(19) United States
(12) Patent Application Publication
(10) Pub. No.: US 2026/0041511 A1
(43) Pub. Date: Feb. 12, 2026

(54) CONTROL SYSTEM FOR A SURGICAL ROBOTIC SYSTEM

(71) Applicant: CMR Surgical Limited, Cambridge (GB)

(72) Inventors: David Christopher Moore, Cambridge (GB); Edward James Wildin Tucker, Cambridge (GB)

(73) Assignee: CMR Surgical Limited, Cambridge (GB)

(21) Appl. No.: 19/150,064

(22) PCT Filed: Feb. 19, 2024
(86) PCT No.: PCT/GB2024/050429

§ 371 Date: Jul. 22, 2025

(30) Foreign Application Priority Data
Feb. 21, 2023 (GB) 2302465.6

(51) Int. Cl. A61B 34/37; A61B 34/30

(52) U.S. Cl. CPC A61B 34/37; A61B 2034/305

(57) ABSTRACT

A control system and a method for controlling a surgical robotic system are provided. The surgical robotic system comprises a surgical robot arm. The surgical robot arm comprises a plurality of joints by which its configuration can be altered. An input is received which indicates a desired motion of the surgical robot arm when the surgical robotic system is operating in a mode in which the surgical robot arm is controlled in accordance with an objective. The objective is to control a particular part of the surgical robot arm to have a desired position and/or orientation. The control system determines that the desired motion of the surgical robot arm would cause a limit to be exceeded. In response to determining that the desired motion of the surgical robot arm would cause the limit to be exceeded, a control signal is generated for controlling the surgical robot arm, and the generated control signal is sent to the surgical robot arm in order to control the surgical robot arm. The control signal is generated, in response to determining that the desired motion of the surgical robot arm would cause the limit to be exceeded, such that: (i) movement of a first set of one or more of the joints of the surgical robot arm is restricted, and (ii) movement of a second set of one or more of the joints of the surgical robot arm is not restricted.

### BACKGROUND

This invention relates to a control system for a surgical robotic system.

It is known to use robots for assisting and performing surgery. FIG. 1 illustrates a surgical robotic system 101. A surgical robot 100 consists of a base 102, a surgical robot arm 104 and a surgical instrument 106 for manipulating tissue. The base supports the robot, and may itself be attached rigidly to, for example, the operating theatre floor, the operating theatre ceiling or a cart. The surgical robot arm extends between the base and the surgical instrument. The surgical robot arm is articulated by means of multiple flexible joints 108 along its length, which are used to alter the configuration of the surgical robot arm to locate the surgical instrument in a desired location relative to a patient. The surgical instrument is attached to the distal end of the surgical robot arm. During a surgical procedure, the surgical instrument may penetrate the body of the patient at a port so as to access the surgical site. The surgical instrument comprises a shaft connected to a distal end effector 110 by a jointed articulation. The end effector performs aspects of a medical procedure, e.g. by engaging in a surgical procedure. This type of medical procedure is often referred to as a minimally invasive surgical procedure. In FIG. 1, the illustrated end effector is a pair of jaws which may be used, for example, for cutting or grasping tissue or as a needle holder.

The configuration of the surgical robot arm 104 may be remotely controlled in response to inputs received at a remote surgeon console 112. A surgeon may provide inputs to the surgeon console. The remote surgeon console may comprise one or more surgeon input devices 114. For example, these may take the form of one or more hand controllers, foot pedals, interactive touch screens etc. A video feed of the surgical site may be captured by an endoscope, often attached to a further surgical robot arm (not shown in FIG. 1 for simplicity), and displayed at a display 116 of the remote surgeon console.

A control system 118 connects the surgeon console 112 to the surgical robot arm 104. The control system receives inputs from the surgeon input device(s) 114 and converts these to control signals to move the joints of the surgical robot arm 104 and end effector 110. The generation of the control signals, based on the inputs, can be performed using inverse kinematics. The control system causes these control signals to be sent to the surgical robot arm in order to control the surgical robot arm. Joint controllers on the robot arm 104 drive the joints 108 to move accordingly.

As well as operating as a master-slave manipulator in which the surgical robot arm 104 is controlled in response to inputs from the surgeon input device(s) 114, in one or more modes of operation the surgical robot arm may be ‘collaborative’ such that a user (e.g. a member of the bedside team) can interact with the surgical robot arm directly by pushing upon it to get it to move. In these modes the surgical robot arm exhibits compliant behaviour, as the control system 118 receives inputs indicating the forces that the user is applying to the surgical robot arm and sends control signals to the surgical robot arm to cause it to move in accordance with the forces that the user is applying to the surgical robot arm.

The surgical robot arm 104 has finite ranges of motion that are possible. For example, each joint 108 of the surgical robot arm 104 may have one or more limits to the way in which it can move. For example, a joint 108 of the surgical robot arm 104 may have a minimum and a maximum angle which it can adopt. A limit for a joint may be a physical limit, e.g. the joint physically cannot move beyond the limit because something physical prevents it from doing so. Alternatively, the limit for a joint may be a software limit, e.g. the control system may be configured not to move a joint beyond its limit even though there might not be anything physically preventing it from doing so. Software limits may be used to prevent (or reduce the likelihood of) the joints adopting positions in which the motion control becomes ambiguous.

Furthermore, there may be a limit on the position of the surgical robot arm for preventing the surgical robot arm from entering a keep out region. A keep out region can be useful for preventing the surgical robot arm from adopting a configuration in which one or more degrees of freedom in the control of the surgical robot arm are lost (e.g. if axes of rotation of two of the joints become collinear) such that the motion control may become ambiguous. Such configurations in which one or more degrees of freedom in the control of the surgical robot arm are lost may be referred to as “singularities”. A keep out region can also be useful for preventing the surgical robot arm from colliding with another component (e.g. another surgical robot arm) in the surgical robotic system and/or for preventing the surgical robot arm from colliding with itself.

When a limit has been reached (e.g. when a joint reaches a limit), full control of the surgical robot arm might not be possible because movement beyond the limit is prevented. As such, when the control system generates the control signals for controlling the surgical robot arm, it attempts to do so in a manner that avoids situations in which limits are reached. However, since the inputs indicating a desired motion of the surgical robot arm 104 (e.g. from the surgeon console and/or due to a user pushing on the surgical robot arm in a compliant mode) cannot be predicted in advance, there are still occasions when a limit may be reached (e.g. when a joint reaches a limit).

An error may occur if an input indicates that the desired motion of the surgical robot arm would cause a limit to be exceeded, and in response to the error any further motion of the surgical robot arm is prevented (i.e. the surgical robot arm is locked) and a user is alerted of the situation. The user (e.g. a member of the bedside team) can respond to the alert by stopping the input which would cause the limit to be exceeded, e.g. by stopping pushing on the surgical robot arm in the direction that has caused the limit to be reached or by stopping the input on the surgeon console that has caused the limit to be reached, such that the surgical robot arm can be unlocked. Then the user can move the surgical robot away from the limit without exceeding the limit, e.g. so that a joint that had reached its limit is no longer at its limit. For kinematically redundant surgical robot arms, this movement of the surgical robot may be performed without moving the position or orientation of the end effector 110 of the surgical instrument.

A limit being reached (e.g. a joint reaching a limit) can effectively mean reducing the number of degrees of freedom that are available for controlling the surgical robot arm. Some objectives for the control of the surgical robot arm (e.g. controlling the instrument tip position and/or orientation or controlling a wrist position and/or orientation) might not be achievable with the reduced number of degrees of freedom. Allowing a user to move the joints in this situation could end up in the objectives being violated. Therefore, in response to a limit being reached, e.g. in response to a joint reaching a limit (and full control of the surgical robot arm being lost), locking the surgical robot arm is a safe thing to do because it prevents the surgical robot arm from moving in an erroneous manner, which has the potential to cause damage to a patient during a surgical procedure.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

There is provided a control system for a surgical robotic system, the surgical robotic system comprising a surgical robot arm, wherein the surgical robot arm comprises a plurality of joints by which its configuration can be altered, wherein the control system is configured to:
receive an input which indicates a desired motion of the surgical robot arm when the surgical robotic system is operating in a mode in which the surgical robot arm is controlled in accordance with an objective, wherein the objective is to control a particular part of the surgical robot arm to have a desired position and/or orientation;
determine that the desired motion of the surgical robot arm would cause a limit to be exceeded; and
in response to determining that the desired motion of the surgical robot arm would cause the limit to be exceeded:
   generate a control signal for controlling the surgical robot arm; and
   cause the generated control signal to be sent to the surgical robot arm in order to control the surgical robot arm;
   wherein the control signal is generated, in response to determining that the desired motion of the surgical robot arm would cause the limit to be exceeded, such that: (i) movement of a first set of one or more of the joints of the surgical robot arm is restricted, and (ii) movement of a second set of one or more of the joints of the surgical robot arm is not restricted.

The limit may be associated with one of the joints of the surgical robot arm, and the control system may be configured to determine that the desired motion of the surgical robot arm would cause the limit to be exceeded by determining that said one of the joints of the surgical robot arm has reached the limit.

Said one of the joints that has reached the limit may be in the first set of one or more of the joints of the surgical robot arm.

The first set of one or more joints may comprise a plurality of joints, and the limit may be associated with at least one, but not all, of the joints of the first set of joints of the surgical robot arm.

The limit may be a limit on the position of the surgical robot arm for preventing the surgical robot arm from entering a keep out region.

The control system may be further configured to define the keep region dynamically based on positions of one or more other components in the surgical robotic system.

The control system may be configured to determine that the desired motion of the surgical robot arm would cause the limit to be exceeded by detecting a clash indicating that the surgical robot arm has reached the edge of the keep out region.

The control signal may be generated, in response to determining that the desired motion of the surgical robot arm would cause the limit to be exceeded, such that: (i) movement of the first set of one or more of the joints of the surgical robot arm is prevented, and (ii) movement of the second set of one or more of the joints of the surgical robot arm is allowed.

The control signal may be generated, in response to determining that the desired motion of the surgical robot arm would cause the limit to be exceeded, such that:
(i) movement of the first set of one or more of the joints of the surgical robot arm is restricted so as to: (a) allow motion of the first set of joints which would cause the surgical robot arm to move away from the limit without exceeding the limit, and (b) prevent motion of the first set of joints which would cause the surgical robot arm to move beyond the limit, and
(ii) movement of the second set of one or more of the joints of the surgical robot arm is not restricted.

The surgical robot arm satisfying the objective may be independent of the angle of each of the one or more joints of the second set.

The surgical robot arm satisfying the objective may be dependent on the angle of each of the one or more joints of the first set.

The particular part of the surgical robot arm may be a wrist of the surgical robot arm.

The surgical robot arm may comprise an attachment for a surgical instrument at a distal end of the surgical robot arm, and a position and/or orientation of a surgical instrument attached to the surgical robot arm might not be a constraint in satisfying the objective.

The mode may be a compliant mode in which the received input which indicates a desired motion of the surgical robot arm is indicative of a force that a user has applied to the surgical robot arm, and the control system may be configured to generate a control signal for controlling the surgical robot arm to move in accordance with the force.

The surgical robot arm may comprise an attachment for a surgical instrument at a distal end of the surgical robot arm, and the mode may be an instrument change mode or an instrument adjust mode in which the control system is configured to control the surgical robot arm such that movement of a surgical instrument attached to the surgical robot arm is constrained to maintain an intersection between the surgical instrument and a pivot point.

The mode may be the instrument change mode, and the control system may be configured to control the surgical robot arm such that movement of a surgical instrument attached to the surgical robot arm is further constrained to being along an axis parallel to a longitudinal axis of a shaft of the surgical instrument.

The surgical robot arm may comprise an attachment for a surgical instrument at a distal end of the surgical robot arm, wherein the most distal joint of the surgical robot arm may be a roll joint that is arranged such that, when a surgical instrument comprising a shaft is attached to the attachment, an axis of rotation of the roll joint is collinear with a longitudinal axis of the shaft of the surgical instrument. The roll joint may be in the second set of one or more joints of the surgical robot arm.

Said roll joint may be the only joint in the second set of one or more joints of the surgical robot arm.

The first set of one or more joints may comprise all of the joints of the surgical robot arm except for said roll joint.

The surgical robot arm may comprise an attachment for a surgical instrument at a distal end of the surgical robot arm, wherein a first plurality of the joints of the surgical robot arm may form a first group of joints which are controllable to control a position of a wrist of the surgical robot arm, and wherein a second plurality of the joints of the surgical robot arm may form a second group of joints which are controllable to control an orientation of the surgical instrument relative to the wrist. If the limit would be exceeded due to desired motion of one of the joints in the first group of joints then the first group of joints may be said first set of one or more joints, and the second group of joints may be said second set of one or more joints. If the limit would be exceeded due to desired motion of one of the joints in the second group of joints then the second group of joints may be said first set of one or more joints, and the first group of joints may be said second set of one or more joints.

The control system may be further configured to determine which of the joints of the surgical robot arm are in the first set and which of the joints of the surgical robot arm are in the second set in dependence upon one or both of: (i) a mode in which the surgical robotic system is operating, and (ii) a current pose of the surgical robot arm.

The one or more joints that are in the first set and the one or more joints that are in the second set may be predetermined.

The control system may be further configured to determine which of the joints of the surgical robot arm are in the first set and which of the joints of the surgical robot arm are in the second set in response to determining that the desired motion of the surgical robot arm would cause the limit to be exceeded.

There is provided a surgical robotic system comprising:
a surgical robot arm comprising a plurality of joints by which its configuration can be altered, the surgical robot arm having an attachment for a surgical instrument at a distal end of the surgical robot arm; and
a control system as described herein.

There is provided a method of controlling a surgical robot arm in a surgical robotic system, wherein the surgical robot arm comprises a plurality of joints by which its configuration can be altered, the method comprising:
receiving an input which indicates a desired motion of the surgical robot arm when the surgical robotic system is operating in a mode in which the surgical robot arm is controlled in accordance with an objective, wherein the objective is to control a particular part of the surgical robot arm to have a desired position and/or orientation;
determining that the desired motion of the surgical robot arm would cause a limit to be exceeded; and
in response to determining that the desired motion of the surgical robot arm would cause the limit to be exceeded:
   generating a control signal for controlling the surgical robot arm; and
   causing the generated control signal to be sent to the surgical robot arm in order to control the surgical robot arm;
   wherein the control signal is generated, in response to determining that the desired motion of the surgical robot arm would cause the limit to be exceeded, such that: (i) movement of a first set of one or more of the joints of the surgical robot arm is restricted, and (ii) movement of a second set of one or more of the joints of the surgical robot arm is not restricted.

There is provided a computer readable storage medium having stored thereon computer readable instructions that, when executed at a control system for a surgical robotic system, cause the control system to perform any of the methods described herein.

The above features may be combined as appropriate, as would be apparent to a skilled person, and may be combined with any of the aspects of the examples described herein.

### BRIEF DESCRIPTION OF THE DRAWINGS

Examples will now be described in detail with reference to the accompanying drawings in which:

FIG. 1 shows an example surgical robotic system;

FIG. 2 shows an example surgical robot arm with a surgical instrument attached;

FIG. 3 shows an exploded view of the joints of the surgical robot arm and the surgical instrument of FIG. 2; and

FIG. 4 is a flow chart for a method of controlling a surgical robot arm using a control system.

The accompanying drawings illustrate various examples. The skilled person will appreciate that the illustrated element boundaries (e.g., boxes, groups of boxes, or other shapes) in the drawings represent one example of the boundaries. It may be that in some examples, one element may be designed as multiple elements or that multiple elements may be designed as one element. Common reference numerals are used throughout the figures, where appropriate, to indicate similar features.

### DETAILED DESCRIPTION

The following description is presented by way of example to enable a person skilled in the art to make and use the invention. The present invention is not limited to the embodiments described herein and various modifications to the disclosed embodiments will be apparent to those skilled in the art.

In examples described herein, the surgical robotic system is operating in a mode in which the surgical robot arm is controlled in accordance with an objective, where the objective is to control a particular part of the surgical robot arm to have a desired position and/or orientation. The particular part of the surgical robot arm may be a wrist of the surgical robot arm (as described in more detail below). The control system receives an input which indicates a desired motion of the surgical robot arm. When the desired motion of the surgical robot arm would cause a limit to be exceeded (e.g. when one of the joints of the surgical robot arm reaches a limit), it might not be necessary to prevent motion of the entire surgical robot arm. For example, there may be functions that a user could carry out without requiring a joint that has reached a limit to be moved further, and those functions can still be permitted. Therefore, in examples described herein, when the desired motion of the surgical robot arm would cause a limit to be exceeded (e.g. when one of the joints of the surgical robot arm reaches a limit), movement of a first set of one or more of the joints of the surgical robot arm is restricted (e.g. the joints of the first set of one or more joints may be locked), whilst movement of a second set of one or more of the joints of the surgical robot arm is permitted, e.g. not restricted. If a joint has reached a limit then that joint is in the first set of one or more joints, i.e. its movement is restricted, e.g. it may be locked. For example, the control system 118 may detect that a joint has reached a limit (e.g. a position in which it cannot move further in a given direction), and in response may prevent the motion of only those joints in the surgical robot arm that would lead to further motion of the joint that has reached the limit in the direction that it cannot move (i.e. restrict or prevent motion of only the joints in the “first set of one or more joints”). This allows some functionality of the surgical robot arm to still be carried out (e.g. it allows movement of the one or more joints in the “second set of one or more joints”) even though one of the joints of the surgical robot arm has reached a limit. As described above, the surgical robotic system is operated in a mode in which the surgical robot arm is controlled in accordance with an objective (or ‘primary function’), where whether the surgical robot arm satisfies the objective is: (i) dependent on the angle of each of the one or more joints of the first set, but (ii) independent of the angle of each of the one or more joints of the second set. For example, the objective may be to control the position of a part of the surgical robot (e.g. the end effector of the instrument, or a “wrist” of the surgical robot arm as described below), wherein the position of the part of the surgical robot arm is (i) dependent on the angle of each of the one or more joints of the first set, but (ii) independent of the angle of each of the one or more joints of the second set. In an example in which one of the joints has reached a limit, in order to satisfy the objective (e.g. in order to control the position of a part of the surgical robot arm or the end effector on the instrument), the angle of the joint that has reached the limit is dependent on the angle of each of the one or more joints of the first set, but is independent of the angle of each of the one or more joints of the second set. Which of the joints of the surgical robot arm are in the first set of one or more joints, and which of the joints of the surgical robot arm are in the second set of one or more joints, may depend on a mode (e.g. surgical mode, compliant mode or instrument retract mode) in which the surgical robotic system is operating, and may also depend on a current pose of the surgical robot arm.

In conventional surgical robotic systems in which all of the joints of the surgical robot arm are locked in response to a limit being reached (e.g. in response to one of the joints reaching a limit), a user might not expect all of the joints to be locked, and may attempt to move one of the joints of the surgical robot arm, expecting compliant behaviour, but the arm will not move (because all of the joints are locked) and the attempt to move it may lead to a tracking error. A tracking error means that the actual position of one or more of the joints of the surgical robot differs from the position commanded by the control system 118. In other words, a tracking error is the difference between the desired position of a joint and the position of the joint measured at the end of the gear train. When a tracking error is larger than a threshold, the surgical robotic system 101 may need to be reset and/or recalibrated in order to proceed with the surgical procedure. As such, a tracking error can be a serious event, which should be avoided if possible. The occurrences of such tracking errors can be reduced in examples described herein because, in response to a limit being reached (e.g. in response to one of the joints reaching a limit), fewer joints are locked (in particular only those joints that need to be locked to ensure safety, i.e. correct operation of the surgical robot, are locked).

As described above, the limit may be associated with one of the joints of the surgical robot arm (e.g. it may be a limit on the angle that the joint can adopt), and determining that the desired motion would cause the limit to be exceeded may involve determining that that one of the joints has reached the limit. In other examples, the limit may be a limit on the position of the surgical robot arm for preventing the surgical robot arm from entering a keep out region. A keep out region may define a region of space which the surgical robot arm should not occupy, e.g. because another component (e.g. another surgical robot arm) in the surgical robotic system is in that region. In this way, a keep out region can be useful for preventing the surgical robot arm from colliding with another component (e.g. another surgical robot arm) in the surgical robotic system and/or for preventing the surgical robot arm from colliding with itself. A keep out region may define a particular configuration of one or more of the joints of the surgical robot arm, e.g. to avoid the surgical robot arm adopting a pose in which one or more degrees of freedom in the control of the surgical robot arm are lost (e.g. if axes of rotation of two of the joints become collinear) such that the motion control of the surgical robot arm may become ambiguous. As mentioned above, such configurations in which one or more degrees of freedom in the control of the surgical robot arm are lost may be referred to as “singularities”. The keep out region may be defined dynamically. A clash may be detected which can indicate that the surgical robot arm has reached the edge of a keep out region. A clash may occur when the surgical robot arm moves into contact with another component (e.g. another surgical robot arm) in the operating room, and may be detected based on torque sensor readings on the surgical robot arm.

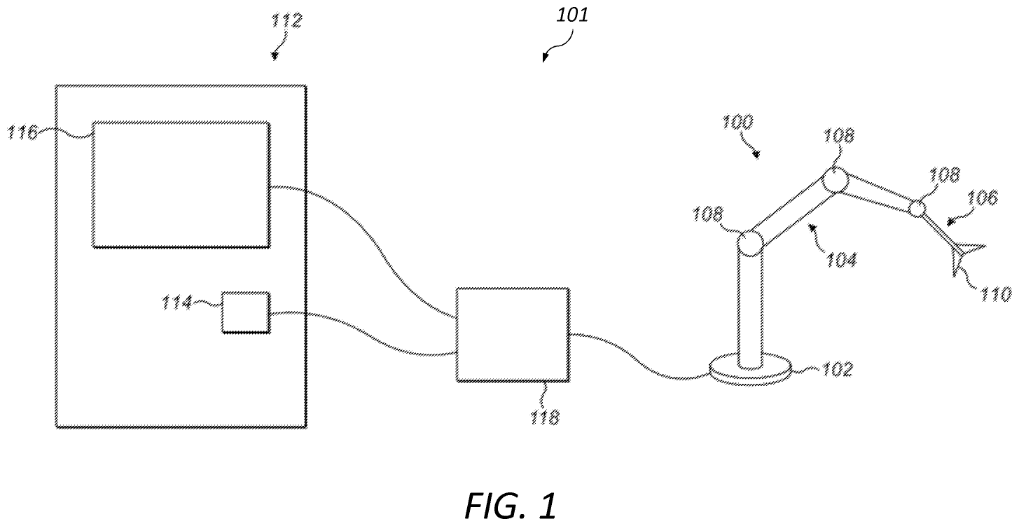

A surgical robotic system of the type illustrated in FIG. 1 is described. As described above, the surgical robotic system 101 comprises one or more surgical robot arm 104 and surgical instrument 106, along with a remote surgeon console 112. The remote surgeon console is connected to the surgical robot arm(s) via a control system 118. The control system may include a central controller located remotely from the surgical robot arm(s), and may also include a robot arm controller per surgical robot arm co-located with that surgical robot arm.

The control system and methods described in the following are done so with respect to a surgical robot arm holding a surgical instrument having an end effector at its distal end for manipulating tissue of a patient at a surgical site. The end effector may be, for example, a pair of jaws, scalpel, suturing needle etc. However, the same surgical robot arm, control system and methods apply equally to a surgical instrument which is an endoscope having a camera at its distal end for capturing a video feed of a surgical site.

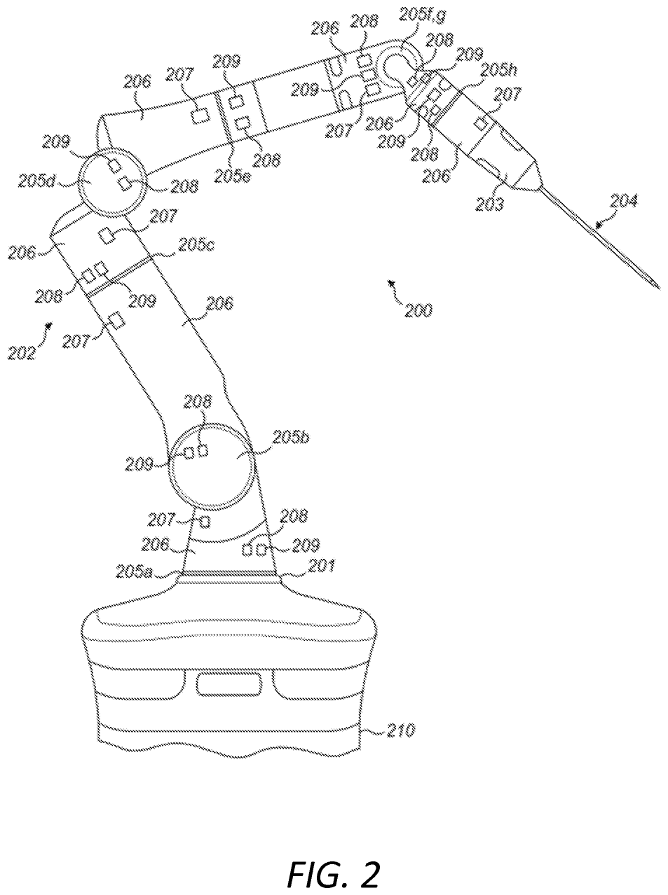

FIG. 2 illustrates an exemplary surgical robot 200, which may be implemented as the surgical robot 100 in the surgical robotic system 101 shown in FIG. 1. The surgical robot 200 comprises a base 201 which is fixed in place when a surgical procedure is being performed. The base 201 may be mounted to a support structure. In FIG. 2, the support structure is a cart 210. This cart may be a bedside cart for mounting the surgical robot at bed height. Alternatively, the support structure may be a ceiling mounted device, or a bed mounted device.

A surgical robot arm 202 extends from the base 201 of the surgical robot to a terminal end 203 which has an attachment for attaching to a surgical instrument 204. The “terminal end” of the surgical robot arm may be referred to as the “distal end” of the surgical robot arm. The surgical robot arm is flexible. It is articulated by means of multiple flexible joints 205 along its length. In between the joints are rigid arm links 206. The joints may be revolute joints. The surgical robot arm has at least seven joints between the base and the terminal end. The surgical robot arm 200 illustrated in FIG. 2 has eight joints in total between the base 201 and the terminal end 203. The surgical robot arm illustrated in FIG. 2 has (only) eight joints between the base and the terminal end. The joints include one or more roll joints (which have an axis of rotation along the longitudinal direction of the arm links on either side of the joint), one or more pitch joints (which have an axis of rotation transverse to the longitudinal direction of the preceding arm link), and one or more yaw joints (which also have an axis of rotation transverse to the longitudinal direction of the preceding arm link and also transverse to the rotation axis of a co-located pitch joint). In the example of FIG. 2: joints 205a, 205c, 205e and 205h are roll joints; joints 205b, 205d and 205f are pitch joints; and joint 205g is a yaw joint. The order of the joints sequentially from the base 201 of the robot arm to the terminal end 203 of the robot arm is: roll, pitch, roll, pitch, roll, pitch, yaw, roll. There are no intervening joints in FIG. 2.

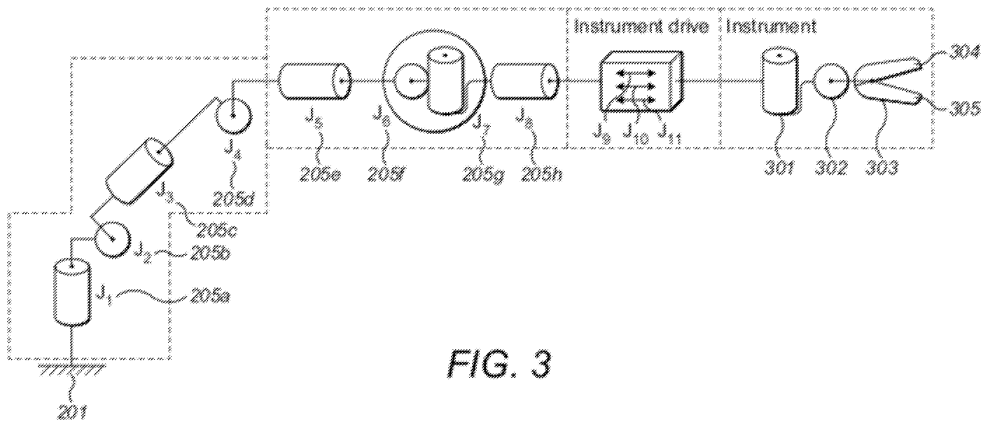

The joints of the surgical robot arm of FIG. 2 are illustrated on FIG. 3. The robot arm is articulated by eight joints. Roll joint J₁ 205a is adjacent to the base 201, and is followed by a pitch joint J₂ 205b. The pitch joint J₂ has a rotation axis perpendicular to the rotation axis of the roll joint J₁. Roll joint J₃ 205c is adjacent to the pitch joint J₂, and is followed by a pitch joint J₄ 205d. The pitch joint J₄ has a rotation axis perpendicular to the rotation axis of the roll joint J₃. Roll joint J₅ 205e is adjacent to the pitch joint J₄, and is followed by a pitch joint J₆ 205f and a yaw joint J₇ 205g, followed by a roll joint J₈ 205h. The pitch joint J₆ and yaw joint J₇ form a compound joint, which may be a spherical joint. The pitch joint J₆ and the yaw joint J₇ have intersecting axes of rotation, which may be perpendicular to each other. The compound joint formed of the pitch joint J₆ and the yaw joint J₇ may be implemented as a “Hooke's” or universal joint. The compound joint formed of the pitch joint J₆ and the yaw joint J₇ may be referred to as a “wrist joint” of the surgical robot arm.

The end of the robot arm distal to the base can be articulated relative to the base by movement of one or more of the joints of the arm. The rotation axes of the set of distal joints J₅, J₆, J₇ and J₈ all intersect at a point on the surgical robot arm. Reference is made to a wrist. The wrist is a portion of the surgical robot arm which rigidly couples to the distal end of a surgical instrument when that surgical instrument is attached to the surgical robot arm. The wrist has a position and an orientation. For example, the position of the wrist (which may be referred to as the “wrist position”) may be the intersection of the rotation axes of J₅, J₆, J₇ and J₈. In other words, the position of the wrist may be the position of the wrist joint, i.e. the position of the compound joint formed by J₆ and J₇. The configuration of the wrist (i.e. the configuration of joints J₅, J₆, J₇ and J₈) define the orientation of the shaft of the surgical instrument extending away from the wrist. The surgical robot arm illustrated in FIGS. 2 and 3 has at least one redundant joint, i.e. it is a kinematically redundant surgical robot arm. In particular, the surgical robot arm illustrated in FIGS. 2 and 3 has eight joints, so in theory two degrees of freedom are redundant. For a given position of the wrist relative to the base of the surgical robot arm, there is more than one configuration of the joints J₁ to J₄. Thus, the surgical robot arm can adopt different poses whilst maintaining the same wrist position.

The surgical robot arm could be jointed differently to that illustrated in FIGS. 2 and 3. For example, the arm may have fewer than eight or more than eight joints. The arm may include joints that permit motion other than rotation between respective sides of the joint, for example a telescopic joint. The arm may have a different ordering of joints to the surgical robot arm depicted in FIGS. 2 and 3.

Returning to FIG. 2, the surgical robot arm comprises a set of motors 207. Each motor 207 drives one or more of the joints 205. Each motor 207 is controlled by a joint controller. The joint controller may be co-located with the motor 207. A joint controller may control one or more of the motors 207. The robot arm comprises a series of sensors 208, 209. These sensors comprise, for each joint, a position sensor 208 for sensing the position of the joint, and a torque sensor 209 for sensing the applied torque about the joint's rotation axis. The torque applied about a joint's rotation axis includes any one or combination of the following components: torque due to gravity acting on the joint, torque due to inertia, and torque due to an external force applied to the joint. One or both of the position and torque sensors for a joint may be integrated with the motor for that joint. The outputs of the sensors are passed to the control system 118.

The surgical instrument 204 attaches to an attachment (e.g. a drive assembly) at the distal end (i.e. the terminal end) 203 of the surgical robot arm 202. During a surgical procedure, this attachment is at all times external to the patient. The surgical instrument 204 has an elongate profile, with a shaft spanning between its proximal end which attaches to the robot arm and its distal end which accesses the surgical site within the patient's body. When the surgical instrument is attached to the attachment of the surgical robot arm, it is configured to extend linearly parallel with the rotation axis of the joint 205h of the arm. For example, the surgical instrument may extend along an axis coincident with the rotation axis of the joint 205h of the arm. In other words, when the surgical instrument is attached to the attachment of the surgical robot arm, the axis of rotation of the roll joint 205h is collinear with a longitudinal axis of the shaft of the surgical instrument.

The proximal end of the surgical instrument and the instrument shaft may be rigid with respect to each other and rigid with respect to the distal end of the robot arm when attached to it. During a surgical procedure, an incision can be made into the patient's body, through which a port can be inserted. The surgical instrument may penetrate the patient's body through the port to access the surgical site. Alternatively, the surgical instrument may penetrate the body through a natural orifice of the body to access the surgical site. At the proximal end of the instrument, the shaft is connected to an instrument interface. The instrument interface engages with the drive assembly at the distal end of the robot arm. Specifically, individual instrument interface elements of the instrument interface each engage a respective individual drive assembly interface element of the drive assembly of the surgical robot arm. The instrument interface is releasably engageable with the drive assembly. The instrument can be detached from the robot arm manually without requiring any tools. This enables the instrument to be detached from the drive assembly quickly and another instrument attached during an operation.

At the distal end of the surgical instrument, the distal end of the instrument shaft is connected to an end effector by an articulated coupling. The end effector engages in a surgical procedure at the surgical site. The end effector may be, for example, a pair of jaws, a pair of monopolar scissors, a needle holder, a fenestrated grasper, or a scalpel. The articulated coupling comprises several joints. These joints enable the pose of the end effector to be altered relative to the direction of the instrument shaft. The end effector itself may also comprise joints. The end effector illustrated in FIGS. 2 and 3 has a pair of opposing end effector elements (or “jaws”) 304, 305. The joints of the end effector are illustrated on FIG. 3 as a pitch joint 301, a yaw joint 302 and a pinch joint 303. It is noted that the naming of joint 301 as a “pitch” joint and the naming of joint 302 as a “yaw” joint (rather than calling joint 301 a yaw joint and calling joint 302 a pitch joint) is an arbitrary choice: the two joints can be rotated by 90 degrees to that shown in FIG. 3 by rotating joint J₈ by 90 degrees. The pitch joint 301 is adjacent to the shaft of the instrument and rotates about an axis perpendicular to the longitudinal axis of the instrument shaft. The yaw joint 302 has a rotation axis perpendicular to the rotation axis of the pitch joint 301. The pinch joint 303 determines the spread of the end effector elements. In practice, the pinch joint 303 may be another yaw joint which has the same rotation axis (or a parallel rotation axis) as the yaw joint 302. Independent operation of the two yaw joints 302, 303 can cause the end effector elements to move in unison, and/or to open and close with respect to each other.

Drive is transmitted from the robot arm to the end effector in any suitable manner. For example, the joints of the instrument may be driven by driving elements such as cables, push rods or push/pull rods. These driving elements engage the instrument interface at the proximal end of the instrument. The drive assembly at the terminal end of the robot arm comprises instrument drive joints which transfer drive from the surgical robot arm to the instrument interface via the respective interface elements described above, and thereby to the instrument joints. These instrument drive joints are shown on FIG. 3 as joints J₉, J₁₀ and J₁₁. FIG. 3 illustrates three instrument drive joints, each one of which drives one of the three joints of the instrument. Movement of the instrument drive joints (J₉, J₁₀ and J₁₁) does not alter the configuration of the surgical robot arm.

The surgeon console 112 is located remotely from the one or more surgical robot arms 202 of the surgical robotic system 101. The surgeon console comprises one or more surgeon input devices 114 and a display 116. Each surgeon input device enables the surgeon to provide a control input to the control system 118. A surgeon input device may, for example, be a hand controller, a foot controller such as a pedal, a touch sensitive input to be controlled by a finger or another part of the body, a voice control input device, an eye control input device or a gesture control input device. The surgeon input device may provide several inputs which the surgeon can individually operate.

For example, the surgeon input device may be a hand controller connected to the surgeon console, for example by a gimbal arrangement. This enables the hand controller to be moved with three degrees of translational freedom with respect to the surgeon console. Such movement may be used to command corresponding movement of the end effector of the instrument. The hand controller may also be rotated with respect to the surgeon console. Such movement may be used to command corresponding rotation of the end effector of the instrument.

The surgeon console may comprise two or more surgeon input devices. Each surgeon input device may be used to control a different surgical instrument. Thus, for example, a surgeon may control one surgical instrument using a hand controller in his left hand, and control another surgical instrument using a hand controller in his right hand.

As described above, the control system 118 connects the surgeon input device 114 to the surgical robot arm 202 of the surgical robot 200. The control system 118 may be separate from the remote surgeon console 112 and the surgical robot 200. The control system 118 may be collocated with the remote surgeon console. The control system 118 may be collocated with the surgical robot 200. The control system 118 may be distributed between the remote surgeon console and the surgical robot 200. The control system comprises a processor and a memory. The memory stores, in a non-transient way, software code that can be executed by the processor to cause the processor to control the surgical robot arm 202 (e.g. by controlling the motors 207 in order to alter the configuration of the surgical robot arm) in the manner described herein.

The control system 118 may receive inputs from the surgeon input device 114 which indicate desired motion of the surgical robot arm (or desired motion of the surgical instrument), and convert those inputs to control signals for moving one or more of the joints 205 of the surgical robot arm 202 in order to alter its configuration. In other situations, the control system 118 may receive inputs (e.g. sensed joint torques) in response to a user pushing on the arm in a compliant mode (i.e. a mode in which the arm exhibits compliant behaviour), where the inputs (e.g. sensed joint torques) indicate desired motion of the surgical robot arm (or desired motion of the surgical instrument), and the control system 118 may process the inputs (e.g. sensed joint torques) to convert them into control signals for moving one or more of the joints 205 of the surgical robot arm 202 in order to alter its configuration. The control signals can be generated by implementing inverse kinematics. In other words, the control signals can be generated based on kinematic equations that define the relationship between the position of the one or more joints 205 and the position of the attachment (at the terminal end 203 of the robot arm 202) for the surgical instrument—as would be well understood by the skilled person. The control system 118 sends these control signals to the surgical robot arm 202, where the corresponding one or more of the joints 205 are driven accordingly. Movement of the surgical instrument 204 attached to the surgical robot arm 202 can thereby be controlled by the control system 118, e.g. in response to movement of the surgeon input device 114.

Constraints may be placed on the movement of the surgical instrument that can be caused by the control system. One such constraint is that the control system 118 is configured to control the surgical robot arm 202, in dependence on inputs received from the surgeon input device 114, to alter the configuration of the surgical robot arm 202 whilst maintaining an intersection between the surgical instrument 204 attached to the surgical robot arm 202 and a pivot point. That is, the control system is configured to control the wrist of the surgical robot arm so that it points towards the pivot point. The control system may be configured to control the surgical robot arm 202 in this way during a minimally invasive procedure when operating in some modes, e.g. a surgical mode or an instrument retract mode. The pivot point may be referred to as a “virtual pivot point” or a “fulcrum”. The pivot point may be mechanically enforced or may be a software constraint enforced by the control system 118. It might be the case that there is nothing physically present at the pivot point (which is why it may be referred to as a virtual pivot point or a virtual fulcrum) and the pivot point is a software constraint enforced by the control system 118 when it determines the control signals for driving the surgical robot arm 202.

As such, the surgical robot arm 202 is driven using control signals which maintain an intersection between the surgical instrument 204 and the pivot point. For example, during the minimally invasive procedure, the surgeon can use the surgeon input device 114 to indicate a desired position and orientation of the end effector of the surgical instrument 204. In response, the control system 118 determines a configuration of the series of joints 205 of the surgical robot arm 202 that will result in both (i) the end effector of the surgical instrument 204 being placed in that desired position and orientation and (ii) the shaft of the surgical instrument 204 passing through (e.g. maintaining an intersection with) the pivot point, and to generate a control signal to move the series of joints 205 to that configuration. By determining a suitable pivot point, the disruption to outer tissues of the patient caused by moving the surgical instrument 204 during a minimally invasive procedure can be minimised. For example, a suitable pivot point may be located at or near a position at which the surgical instrument penetrates the body of the patient (e.g. at a port or natural orifice) so as to access the surgical site. A calibration process can be performed prior to performing a minimally invasive procedure in order to determine a suitable pivot point. The control system 118 can store the pivot point in memory for later use.

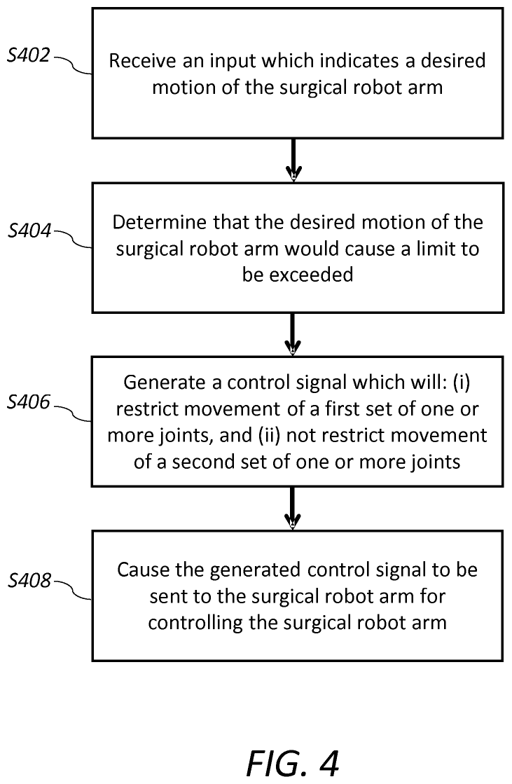

FIG. 4 is a flow chart for a method by which the control system 118 controls the surgical robot arm 202 when the surgical robotic system is operating in a mode in which the surgical robot arm is controlled in accordance with an objective, wherein the objective is to control a particular part (e.g. the wrist) of the surgical robot arm to have a desired position and/or orientation. In step S402 the control system 118 receives an input (e.g. from the surgeon input device 114 or from the torque sensors on the surgical robot arm) which indicates a desired motion of the surgical robot arm 202. As described above, during normal operation of the surgical robotic system 101, the control system 118 generates control signals for controlling the surgical robot arm 202 and sends the generated control signals to the surgical robot arm in order to control the surgical robot arm.

In step S404 the control system 118 determines that the desired motion of the surgical robot arm 202 would cause a limit to be exceeded. For example, in step S404 the control system 118 may determine that one of the joints 205 of the surgical robot arm 202 has reached a limit. In this way step S404 involves detecting a condition indicating that movement of one or more of the joints 205 is to be restricted. Each joint may be associated with one or more limits. The limit may be any type of limit to the movement of the joint. For example, it may be a limit on the angle (e.g. a minimum angle or a maximum angle) which can be adopted by the joint. As described above, the limit for the joint may be a physical limit, e.g. the joint physically cannot move beyond the limit because something physical prevents it from doing so. Alternatively, the limit for the joint may be a software limit, e.g. the control system 118 may be configured not to move the joint beyond its limit even though there might not be anything physically preventing it from doing so. Typically a software limit would be reached before a physical limit is reached. In order to determine that one of the joints 205 has reached a limit, the control system 118 may receive a joint position indication for the joint from the position sensor 208 for that joint, and may determine that the joint position indication indicates that the position of the joint has reached a threshold corresponding to the limit.

In examples described in the preceding paragraph, the limit is associated with a joint. In other examples, the limit may be associated with the pose of the surgical robot arm (e.g. for preventing the surgical robot arm from entering a keep out region), or the limit may be associated with collisions with the other components in the environment (including other robot arms, operators, and other equipment in an operating room). As mentioned above, a collision may be detected as a ‘clash’ which indicates that the surgical robot arm has reached the edge of a keep out region, and may be detected based on monitoring joint torques, e.g. to detect a sudden increase in a joint torque.

In response to determining, in step S404, that the desired motion of the surgical robot arm 202 would cause a limit to be exceeded, the control system 118 performs steps S406 and S408. In step S406 the control system 118 generates a control signal for controlling the surgical robot arm. In step S408 the control system 118 causes the generated control signal to be sent to the surgical robot arm 202 in order to control the surgical robot arm. The control signal is generated in step S406, in response to determining in step S404 that the desired motion of the surgical robot arm 202 would cause a limit to be exceeded (e.g. in response to determining that one of the joints has reached the limit), such that: (i) movement of a first set of one or more of the joints of the surgical robot arm is restricted (e.g. prevented such that the joint(s) of the first set of one or more joints are locked), and (ii) movement of a second set of one or more of the joints of the surgical robot arm is allowed (e.g. not restricted). For example, in response to determining that the desired motion of the surgical robot arm 202 would cause a limit to be exceeded (e.g. in response to determining that one of the joints has reached the limit), movement of the first set of one or more of the joints of the surgical robot arm is not permitted, but movement of the second set of one or more of the joints of the surgical robot arm is permitted. If it is determined in step S404 that a joint has reached a limit then the joint that has reached the limit is in the first set of one or more of the joints of the surgical robot arm, e.g. such that that joint is locked. As described above, allowing one or more of the joints (i.e. the joint(s) in the second set) to move after a different one of the joints (i.e. a joint in the first set) has reached a limit means that some functionality of the surgical robot arm is still available. In particular, this can lead to a reduction in tracking errors if a user attempts to implement that functionality after the joint has reached the limit.

In some examples, in response to determining that the desired motion of the surgical robot arm 202 would cause a limit to be exceeded (e.g. in response to determining that one of the joints has reached the limit) movement of the first set of one or more of the joints of the surgical robot arm is prevented (i.e. the one or more joints of the first set are locked), and movement of the second set of one or more of the joints of the surgical robot arm is allowed.

In other examples, some movement of the one or more joints of the first set is allowed, but only if that movement would not push the surgical robot arm further towards the limit (i.e. only if that movement would not push the surgical robot arm such that the limit is exceeded). That is, in these other examples, in response to determining that the desired motion of the surgical robot arm 202 would cause a limit to be exceeded (e.g. in response to determining that one of the joints has reached the limit):
(i) movement of the first set of one or more of the joints of the surgical robot arm is restricted so as to: (a) allow motion of the first set of joints which would cause the surgical robot arm to move away from the limit without exceeding the limit (e.g. which would cause a joint that has reached a limit to back away from the limit), and (b) prevent motion of the first set of joints which would cause the surgical robot arm to move beyond the limit (e.g. which would drive a joint that has reached a limit beyond the limit), and
(ii) movement of the second set of one or more of the joints of the surgical robot arm is not restricted.

It can be seen that in response to determining that the desired motion of the surgical robot arm 202 would cause a limit to be exceeded, two sections of the surgical robot arm are controlled to behave differently. The two sections are defined respectively by the first and second sets of one or more joints of the surgical robot arm.

In this way, if a joint reaches its limit and is still being driven towards its limit (i.e. so that the desired motion of the joint is to exceed the limit), one or more of the joints of the surgical robot arm (i.e. the one or more joints of the second set) can still be moved (without restriction). For example, a joint of the first set of one or more joints may be driven towards, and may reach, its limit by a user pushing on a section of the arm in a compliant mode, whilst another part of the surgical robot arm, comprising the one or more joints of the second set, is being manipulated.

As described above, the surgical robotic system 101 is configured to be operated in a mode in which the surgical robot arm 202 is controlled in accordance with an objective (or primary function). For example, the objective may be to control a particular part of the surgical robot to have a desired position and/or orientation. In examples described herein, the surgical robot arm satisfying the objective is independent of the angle of each of the one or more joints of the second set. Therefore, the one or more joints of the second set can be moved, and the objective (i.e. the primary function) can be satisfied, e.g. without needing to move a joint that has reached its limit. Furthermore, in examples described herein, the surgical robot arm satisfying the objective is dependent on the angle of each of the one or more joints of the first set. Therefore, the one or more joints of the first set may be locked, e.g. so that the objective can be satisfied without needing to move a joint that has reached its limit.

In some examples, the ‘particular part’ of the surgical robot may be the end effector of the surgical instrument attached to the surgical robot arm.

In other examples, the ‘particular part’ of the surgical robot may be the wrist of the surgical robot arm (e.g. the part defined by joints J₅, J₆, J₇ and J₈). As described above, the position (or “origin”) of the wrist is defined to be the point where the axes of the J₅, J₆, J₇ and J₈ joints intersect. The orientation of the wrist points from the wrist origin in the direction of the axis of the J₈ joint. As described above, the wrist includes the wrist joint (e.g. the compound joint formed of joints J₆ and J₇ in the example shown in FIGS. 2 and 3) of the surgical robot arm. The surgical robotic system may be configured to be operated in a mode (e.g. a compliant mode or a surgical mode) in which the particular part (e.g. the wrist) of the surgical robot arm is controlled to have a desired position and/or orientation, wherein the desired position and/or orientation of the particular part of the surgical robot arm is: (i) independent of the angle of each of the one or more joints of the second set, and (ii) dependent on the angle of each of the one or more joints of the first set. The first set may include a joint that has reached its limit.

As described above, in compliant modes, the surgical robotic system exhibits compliant behaviour such that the surgical robot arm responds when a user pushes on it. Three types of compliant mode are: (i) an instrument change mode (which may be referred to as an instrument retract mode) in which the wrist position is constrained to move along the instrument axis and the wrist pose is constrained in two degrees of freedom, leaving the roll axis free; (ii) an instrument adjust mode in which the wrist position can be moved freely but the pose is constrained in two degrees of freedom, leaving the roll axis free, so that the axis of the J₈ joint points towards the pivot point; and (iii) a port training mode in which the wrist position is constrained to move in a spherical surface with centre at a first approximation of the pivot point whilst the pose is only physically constrained by the port and instrument interaction. In a surgical mode the inverse kinematics implemented by the control system attempts to achieve the wrist pose commanded by the surgeon console. In the instrument change mode, the instrument adjust mode, the port training mode and the surgical mode, the wrist pose (i.e. position and/or orientation) is controlled. In some other modes the wrist pose is not controlled, e.g. in an unlocked mode each joint moves in response to the torques applied to it locally.

In some modes (e.g. an instrument change mode, an instrument adjust mode and a port training mode), a position and/or orientation of the surgical instrument 204 attached to the surgical robot arm 202 is not a constraint in satisfying the objective (or ‘primary function’). In particular, in these modes the position and/or orientation of the end effector of the surgical instrument is not a constraint in satisfying the objective (or ‘primary function’).

When the surgical robot arm is in a compliant mode (which is a mode in which the surgical robot arm exhibits compliant behaviour), the control system 118 drives the arm in dependence on an external force acting on the surgical robot arm 202. For example, the control system is able to cause the arm to move in response to a member of the bedside team pushing on a part of the arm, thereby giving the impression that the member of the bedside team is physically moving the arm. In this way, in the compliant mode the control system is configured to receive an input indicative of a force that a user has applied to the surgical robot arm, and generate a control signal for controlling the surgical robot arm to move in accordance with the force. This functionality is useful in a number of scenarios including those in which a member of the bedside team is changing the instrument connected to the arm and those in which a member of the bedside team notices that a portion of the arm is going to collide with another piece of apparatus in the operating room. In this example, the member of the bedside team is able to push on a part of the robot arm so that the arm moves out of the way of the neighbouring apparatus. As described above, the surgical robot arm is a kinematically redundant surgical robot arm, so parts of it can move without necessarily moving other parts of it. The process of driving the arm in response to an external force being exerted on a part of the arm broadly involves gathering information about the external force which has been imparted on the arm and deciding, based on that force, how the arm should behave in response to that force. How the arm should behave in response to the force is dictated by a chosen impedance model, the properties of which may be chosen arbitrarily to achieve whatever behaviour of the arm is desired.

The instrument change mode (which may be referred to as an instrument retract mode) can be used in order to retract the surgical instrument 204 from a patient's body. For example, the surgical instrument may be retracted from the patient's body after an invasive procedure has been completed or during the procedure. For example, the surgical instrument attached to the surgical robot arm may be changed or swapped during the invasive procedure. The surgical instrument may be swapped in order to use a different surgical instrument having different capabilities, or it may be swapped in the event that the surgical instrument attached to the robot arm is faulty.

In the instrument retract mode, the control system 118 may cause the surgical robot arm 202 to exhibit compliant-like behaviour. The control system may enable such compliant like behaviour so that a member of the bedside team can retract the surgical instrument from the patient's body. The control system may cause the configuration of the robot arm to be altered in response to an externally applied force or torque (e.g. a manual push or pull applied by a member of the operating room staff) so as to enable the surgical instrument to be retracted from the patient's body. In the instrument retract mode (and in an instrument adjust mode), the control system 118 controls the surgical robot arm 202 such that movement of the surgical instrument 204 attached to the surgical robot arm is constrained to maintain an intersection between the surgical instrument and the virtual pivot point. Furthermore, in the instrument retract mode (but not in the instrument adjust mode), the control system 118 controls the surgical robot arm 202 such that movement of the surgical instrument 204 attached to the surgical robot arm is constrained to being along an axis parallel to the longitudinal axis of the shaft of the surgical instrument. In this way, the longitudinal axis of the shaft of the surgical instrument maintains the intersection with the virtual pivot point during the retraction of the surgical instrument in the instrument retract mode. Retracting the surgical instrument from the patient's body along an axis parallel to the longitudinal axis of the surgical instrument minimises or negates damage or disruption to the surrounding tissues of the patient as the instrument is retracted.

In examples described herein, the most distal joint of the surgical robot arm (e.g. joint J₈ in the example shown in FIGS. 2 and 3) is a roll joint that is arranged such that, when the surgical instrument 204 is attached to the attachment at the terminal end 203 of the surgical robot arm 202, an axis of rotation of the roll joint (J₈) is collinear with a longitudinal axis of the shaft of the surgical instrument. In some particularly useful examples, the roll joint (J₈) is in the second set of one or more joints of the surgical robot arm. In particular, the roll joint (J₈) may be the only joint in the second set of one or more joints of the surgical robot arm, and all of the other joints of the surgical robot arm (i.e. joints J₁ to J₇ in the example shown in FIGS. 2 and 3) may be in the first set of one or more joints of the surgical robot arm. In other words, if any of joints J₁ to J₇ reach a limit then all seven of those joints may be locked, but movement of joint J₈ is still allowed. This is particularly useful when the surgical robotic system is operating in a compliant mode in which compliant behaviour of the surgical robot arm is expected.

In conventional systems in which the whole surgical robot arm is locked in response to one of the joints reaching a limit, many tracking errors occur when the surgical robotic system is operating in a mode where J₈ is normally compliant, but became unexpectedly non-compliant due to one of the other joints reaching a limit. The tracking errors often occur when a user attempts to move the J₈ joint, expecting compliant behaviour, but the J₈ joint is locked. One situation in which this can occur is when the surgical instrument is being changed, in which the system is operating in an instrument change mode (or “instrument retract mode”). Under normal circumstances the J₈ joint is compliant in this mode, but as the surgical instrument is retracted away from the patient, one of the joints other than the J₈ joint (e.g. the J₇ joint) may reach a limit, which would, conventionally, lock all of the joints of the surgical robot arm (including joint J₈). Then the user may attempt to twist joint J₈ to change the instrument, which can lead to the tracking error. As an example, these tracking errors may account for approximately 10% of all of the errors that are experienced in conventional surgical robotic systems. However, these tracking errors are avoided in examples described herein by allowing J₈ to move even when one of the other joints has reached a limit. In particular, joint J₈ can remain compliant in modes where it is usually compliant, even if other parts of the surgical robot arm are locked following one of the joints (other than joint J₈) reaching a limit. This will significantly reduce the occurrences of tracking errors in the surgical robotic system. Furthermore, allowing joint J₈ to move allows a member of the bed side team to change the surgical instrument even on an arm in which another joint has reached a limit. In these examples, compliant movement of joint J₈ is still allowed when another joint reaches a limit because movement of joint J₈ in this case does not require the joint that has reached the limit to make any further movement whilst still satisfying the objective of controlling the position of the wrist of the surgical robot arm. Specifically, joint J₈, which effects a purely rotating movement around the instrument axis can be moved—as it is the most distal joint in the surgical robot arm and only rotates about an axis that is collinear with the longitudinal axis of the shaft of the surgical instrument—without any other joints needing to be moved.

As described above, which of the joints of the surgical robot arm are in the first set of one or more joints, and which of the joints of the surgical robot arm are in the second set of one or more joints, may depend on a mode (e.g. surgical mode, instrument change mode, instrument adjust mode, etc.) in which the surgical robotic system is operating. The one or more joints that are in the first set and the one or more joints that are in the second set may be predetermined. For example, when the surgical robotic system is operating in a mode in which compliant behaviour is expected, as described in the preceding two paragraphs, it may be predetermined that the most distal joint (J₈) is the only joint in the second set of joints and all of the other joints (J₁ to J₇) are in the first set of joints. In this example, the J₈ joint is known to be independent of the other joints in the surgical robot arm because it is the final joint (i.e. the most distal joint) in the surgical robot arm and its axis of rotation coincides with the longitudinal axis of the shaft of the surgical instrument.

Additionally or alternatively (to depending on the mode in which the surgical robotic system is operating), which of the joints of the surgical robot arm are in the first set of one or more joints, and which of the joints of the surgical robot arm are in the second set of one or more joints may depend on a current pose of the surgical robot arm. As such, in response to determining that the desired motion of the surgical robot arm would cause a limit to be exceeded (e.g. in response to determining that said one of the joints has reached the limit) in step S404, the control system 118 may perform a step of determining which of the joints of the surgical robot arm are in the first set (such that movement of those joints is not permitted) and which of the joints of the surgical robot arm are in the second set (such that movement of those joints is permitted), e.g. based on the current pose of the surgical robot arm.

In general, there may be joints or groups of joints in a kinematic chain (i.e. in the surgical robot arm) that are independent from other joints or groups of joints. For example, the surgical robot arm may use some joints to perform one function and other joints to perform some other independent function. This “independence” may be pose-specific and need to be determined in response to determining that the desired motion of the surgical robot arm would cause a limit to be exceeded; but it may be permanent. In the example described above, in modes in which compliant behaviour is expected and the primary function is controlling the position and orientation of the wrist of the surgical robot arm, joint J₈ is independent of the other joints of the surgical robot arm. As described above, the wrist includes a ‘wrist joint’ in the example shown in FIGS. 2 and 3 which is formed of joints J₆ and J₇.

As another example, a first plurality of the joints of the surgical robot arm (e.g. joints J₁ to J₄) form a first group of joints which are controllable to control a position of the wrist of the surgical robot arm. The position of the wrist (which is where the axes of the J₅, J₆, J₇ and J₈ joints intersect) may be defined with three spatial coordinates, e.g. x, y and z coordinates. Furthermore, a second plurality of the joints of the surgical robot arm (e.g. joints J₅ to J₈) form a second group of joints which are controllable to control an orientation of the surgical instrument relative to the wrist (i.e. the orientation of the wrist). In this example, if a limit would be exceeded due to desired motion of one of the joints in the first group of joints (J₁ to J₄) then the first group of joints is the “first set of one or more joints” described above for which movement is restricted (e.g. the joints of the first group of joints, J₁ to J₄, may be locked), whereas the second group of joints is the “second set of one or more joints” described above for which movement is not restricted (e.g. the joints of the second group of joints, J₅ to J₈, are allowed to move). This means that the function of controlling the position of the wrist may be disabled, but the function of controlling the orientation of the surgical instrument relative to the wrist is not disabled, i.e. some functionality is still enabled. Similarly, if a limit would be exceeded due to desired motion of one of the joints in the second group of joints (J₅ to J₈) then the second group of joints is the “first set of one or more joints” described above for which movement is restricted (e.g. the joints of the second group of joints, J₅ to J₈, may be locked), whereas the first group of joints is the “second set of one or more joints” described above for which movement is not restricted (e.g. the joints of the first group of joints, J₁ to J₄, are allowed to move). This means that the function of controlling the orientation of the surgical instrument relative to the wrist may be disabled, but the function of controlling the position of the wrist is not disabled, i.e. some functionality is still enabled.

The main examples described with reference to FIG. 4 relate to when one of the joints reaches a limit. However, as described above, the limit may be a limit on the position of the surgical robot arm for preventing the surgical robot arm from entering a keep out region. Furthermore, the keep region may be defined dynamically based on positions of one or more other components (e.g. other surgical robot arms) in the surgical robotic system. The control system may be used to control multiple surgical robot arms in the surgical robotic system, so it has knowledge of the positions of the multiple surgical robot arms, which it can use to dynamically define keep regions for the surgical robot arms to therefore avoid collisions between the different surgical robot arms. Further as described above, step S404 of determining that the desired motion of the surgical robot arm would cause a limit to be exceeded may comprise detecting a clash indicating that the surgical robot arm has reached the edge of a keep out region.

As is known in the art, the control system can implement inverse kinematics to determine a set of joint angles for the joints of the surgical robot arm that will achieve the desired objective (e.g. achieve a particular position and/or orientation of a particular part of the surgical robot). As described above, the particular part of the surgical robot may for example be the wrist of the surgical robot arm or the end effector of the surgical instrument. The implementation of the inverse kinematics may use a Jacobian matrix to link the joint angles to the desired position and/or orientation of a particular part of the surgical robot. A “null space” can be defined as being a space that is orthogonal to the Jacobian matrix. Any motions of the surgical robot arm that are in the null space do not affect the position and/or orientation of the particular part of the surgical robot that is being controlled. As such, motions of the surgical robot arm that are purely in the null space are allowable even when a limit has been reached (which would conventionally cause the whole surgical robot arm to be locked). As described above, any motion that is purely in the null space is allowable when a limit has been reached, and this motion may be formed of movements of one joint or a combination of the joints of the surgical robot arm. If the mode changes and a different part of the surgical robot is then being controlled (e.g. if there is a switch from controlling the position and/or orientation of the wrist of the surgical robot arm to controlling the position and/or orientation of the end effector of the surgical instrument) then a different Jacobian matrix will be defined to link the joint angles to the desired position and/or orientation of the particular part of the surgical robot being controlled; and as such the null space will be different. Therefore different motions (e.g. motions of different joints) will be purely in the null space and therefore allowable when a limit has been reached. Furthermore, any motion of joints which come after (i.e. are more distal on the surgical robot arm than) the particular part of the surgical robot arm that is being controlled is independent of the objective of controlling the particular part of the surgical robot arm. For example, if the position of the wrist is being controlled then motions of joints J₅ to J₈ are allowable, e.g. even if one of J₁ to J₄ reaches a limit. As another example, if the position and orientation of the wrist is being controlled then motion of joint J₈ is allowable, e.g. even if one of J₁ to J₇ reaches a limit.

The methods described herein may be implemented by executing computer program code at the control system 118. That is, the control system 118 may comprise a computer readable storage medium having stored thereon computer readable instructions that, when executed on a processing unit at the control system 118, cause the control system to perform any of the methods described herein.

It is to be understood that the robot arm described herein could be for purposes other than surgery. For example, the surgical robot arm could be controlled for manipulating tissue, which is not part of a patient, e.g. for manipulating tissue of a cadaver or of any other object. As another example, the robot arm could control a viewing instrument for viewing inside a manufactured article such as a car engine, via an inspection port.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A control system for a surgical robotic system, the surgical robotic system comprising a surgical robot arm, wherein the surgical robot arm comprises a plurality of joints by which its configuration can be altered, wherein the control system is configured to:
receive an input which indicates a desired motion of the surgical robot arm when the surgical robotic system is operating in a mode in which the surgical robot arm is controlled in accordance with an objective, wherein the objective is to control a particular part of the surgical robot arm to have a desired position and/or orientation;
determine that the desired motion of the surgical robot arm would cause a limit to be exceeded; and
in response to determining that the desired motion of the surgical robot arm would cause the limit to be exceeded:
generate a control signal for controlling the surgical robot arm; and
cause the generated control signal to be sent to the surgical robot arm in order to control the surgical robot arm;
wherein the control signal is generated, in response to determining that the desired motion of the surgical robot arm would cause the limit to be exceeded, such that: (i) movement of a first set of the joints of the surgical robot arm is restricted, and (ii) movement of a second set of one or more of the joints of the surgical robot arm is not restricted, wherein the first set of the joints comprises a plurality of joints, and wherein the limit is associated with at least one, but not all, of the joints of the first set of joints of the surgical robot arm.

2. The control system of claim 1 wherein the limit is associated with one of the joints of the surgical robot arm, and wherein the control system is configured to determine that the desired motion of the surgical robot arm would cause the limit to be exceeded by determining that said one of the joints of the surgical robot arm has reached the limit.

3. The control system of claim 2 wherein said one of the joints that has reached the limit is in the first set of the joints of the surgical robot arm.

4. The control system of claim 1 wherein the surgical robot arm comprises an attachment for a surgical instrument at a distal end of the surgical robot arm, wherein the most distal joint of the surgical robot arm is a roll joint that is arranged such that, when a surgical instrument comprising a shaft is attached to the attachment, an axis of rotation of the roll joint is collinear with a longitudinal axis of the shaft of the surgical instrument, and wherein the roll joint is in the second set of one or more joints of the surgical robot arm.

5. The control system of claim 4 wherein said roll joint is the only joint in the second set of one or more joints of the surgical robot arm.

6. The control system of claim 4 wherein the first set of joints comprises all of the joints of the surgical robot arm except for said roll joint.

7. The control system of claim 1 wherein the limit is a limit on the position of the surgical robot arm for preventing the surgical robot arm from entering a keep out region.

8. The control system of claim 7 further configured to define the keep out region dynamically based on positions of one or more other components in the surgical robotic system.

9. The control system of claim 7 configured to determine that the desired motion of the surgical robot arm would cause the limit to be exceeded by detecting a clash indicating that the surgical robot arm has reached the edge of the keep out region.

10. The control system of claim 1 wherein the control signal is generated, in response to determining that the desired motion of the surgical robot arm would cause the limit to be exceeded, such that: (i) movement of the first set of the joints of the surgical robot arm is prevented, and (ii) movement of the second set of one or more of the joints of the surgical robot arm is allowed.

11. The control system of claim 1 wherein the control signal is generated, in response to determining that the desired motion of the surgical robot arm would cause the limit to be exceeded, such that:
(i) movement of the first set of the joints of the surgical robot arm is restricted so as to: (a) allow motion of the first set of joints which would cause the surgical robot arm to move away from the limit without exceeding the limit, and (b) prevent motion of the first set of joints which would cause the surgical robot arm to move beyond the limit, and
(ii) movement of the second set of one or more of the joints of the surgical robot arm is not restricted.

12. The control system of claim 1 wherein the surgical robot arm satisfying the objective is independent of the angle of each of the one or more joints of the second set.

13. (canceled)

14. The control system of claim 1 wherein the particular part of the surgical robot arm is a wrist of the surgical robot arm.

15. (canceled)

16. The control system of claim 1 wherein the mode is a compliant mode in which the received input which indicates a desired motion of the surgical robot arm is indicative of a force that a user has applied to the surgical robot arm, and wherein the control system is configured to generate a control signal for controlling the surgical robot arm to move in accordance with the force.

17. The control system of claim 16 wherein the surgical robot arm comprises an attachment for a surgical instrument at a distal end of the surgical robot arm, and wherein the mode is an instrument change mode or an instrument adjust mode in which the control system is configured to control the surgical robot arm such that movement of a surgical instrument attached to the surgical robot arm is constrained to maintain an intersection between the surgical instrument and a pivot point.

18. (canceled)

19. The control system of claim 1 wherein the surgical robot arm comprises an attachment for a surgical instrument at a distal end of the surgical robot arm, wherein a first plurality of the joints of the surgical robot arm form a first group of joints which are controllable to control a position of a wrist of the surgical robot arm, and wherein a second plurality of the joints of the surgical robot arm form a second group of joints which are controllable to control an orientation of the surgical instrument relative to the wrist,
wherein if the limit would be exceeded due to desired motion of one of the joints in the first group of joints then the first group of joints is said first set of joints, and the second group of joints is said second set of one or more joints, and
wherein if the limit would be exceeded due to desired motion of one of the joints in the second group of joints then the second group of joints is said first set of joints, and the first group of joints is said second set of one or more joints.

20. The control system of claim 1 further configured to determine which of the joints of the surgical robot arm are in the first set and which of the joints of the surgical robot arm are in the second set in dependence upon one or both of: (i) a mode in which the surgical robotic system is operating, and (ii) a current pose of the surgical robot arm.

21. (canceled)

22. (canceled)

23. A surgical robotic system comprising:
a surgical robot arm comprising a plurality of joints by which its configuration can be altered, the surgical robot arm having an attachment for a surgical instrument at a distal end of the surgical robot arm; and
a control system as claimed in claim 1.

24. A method of controlling a surgical robot arm in a surgical robotic system, wherein the surgical robot arm comprises a plurality of joints by which its configuration can be altered, the method comprising:
receiving an input which indicates a desired motion of the surgical robot arm when the surgical robotic system is operating in a mode in which the surgical robot arm is controlled in accordance with an objective, wherein the objective is to control a particular part of the surgical robot arm to have a desired position and/or orientation;
determining that the desired motion of the surgical robot arm would cause a limit to be exceeded; and
in response to determining that the desired motion of the surgical robot arm would cause the limit to be exceeded:
generating a control signal for controlling the surgical robot arm; and
causing the generated control signal to be sent to the surgical robot arm in order to control the surgical robot arm;
wherein the control signal is generated, in response to determining that the desired motion of the surgical robot arm would cause the limit to be exceeded, such that: (i) movement of a first set of the joints of the surgical robot arm is restricted, and (ii) movement of a second set of one or more of the joints of the surgical robot arm is not restricted, wherein the first set of the joints comprises a plurality of joints, and wherein the limit is associated with at least one, but not all, of the joints of the first set of joints of the surgical robot arm.

25. A computer readable storage medium having stored thereon computer readable instructions that, when executed at a control system for a surgical robotic system, cause the control system to perform the method of claim 24.

26. (canceled)
